SEQUENCE LISTING (44 sequences)

SEQ ID NO: 1 - DNA, 768 bp, Human papillomavirus
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttctgttgcaagtgtgactctacgcttcggctgtgcgtacaaagcacacacgtagacattcgtacgttggaagacctgttaatgggcacactaggaattgtgtgccccatctgttctcagaaaccaatgcaccaaaagagaactgcaatgtttcaggacccacaggagcgacccagaaagttaccacagttatgcacagagctgcaaacaactatacatgatataatattggaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctg

SEQ ID NO: 2 - protein, 256 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 3 - DNA, 60 bp, Foot-and-mouth disease virus
cagctgttgaattttgaccttcttaagcttgcgggagacgtcgagtccaaccctggcccc

SEQ ID NO: 4 - DNA, 2328 bp, Dengue virus
atgaataaccaacgaaaaaaggcgagaaatacccctttcaatatgctgaaacgcgagagaaaccgcgtgtcgactgtacaacagctgacaaagagattctcacttggaatgctgcagggacgaggaccattaaaactgttcatggccctggtggcgttccttcgtttcctaacaatcccaccaacagcagggatactgaagagatggggaacaattaaaaaatcaaaagccattaatgttttgagagggttcaggaaagagattggaaggatgctgaacatcttgaacaggagacgcagaactgcaggcatgatcattatgctgattccaacagtgatggcgttccatttaaccacacgtaacggagaaccacacatgatcgtcagtagacaagagaaagggaaaagtcttctgtttaaaacagaggatggtgtgaacatgtgtaccctcatggccatggaccttggtgaattgtgtgaagatacaatcacgtacaagtgtccttttctcaggcagaatgaaccagaagacatagattgttggtgcaactctacgtccacatgggtaacttatgggacgtgtaccaccacaggagaacacagaagagaaaaaagatcagtggcactcgttccacatgtgggaatgggactggagacacgaactgaaacatggatgtcatcagaaggggcctggaaacatgcccagagaattgaaacttggatcttgagacatccaggctttaccataatggcagcaatcctggcatacaccataggaacgacacatttccaaagagccctgattttcatcttactgacagctgtcgctccttcaatgacaatgcgttgcataggaatatcaaatagagactttgtagaaggggtttcaggaggaagctgggttgacatagtcttagaacatggaagctgtgtgacgacgatggcaaaaaacaaaccaacattggattttgaactgataaaaacagaagccaaacaacctgccactctaaggaagtactgtatagaggcaaagctgaccaacacaacaacagattctcgctgcccaacacaaggagaacccagcctaaatgaagagcaggacaaaaggttcgtctgcaaacactccatggtggacagaggatggggaaatggatgtggattatttggaaaaggaggcattgtgacctgtgctatgttcacatgcaaaaagaacatgaaaggaaaagtcgtgcaaccagaaaacttggaatacaccattgtgataacacctcactcaggggaagagcatgcagtcggaaatgacacaggaaaacatggcaaggaaatcaaaataacaccacagagttccatcacagaagcagagttgacaggctatggcactgtcacgatggagtgctctccgagaacgggcctcgacttcaatgagatggtgttgctgcaaatggaaaataaagcttggctggtgcacaggcaatggttcctagacctgccgttgccatggctgcccggagcggacacacaaggatcaaattggatacagaaagagacattggtcactttcaaaaatccccatgcgaagaaacaggatgttgttgttttgggatcccaagaaggggccatgcacacagcactcacaggggccacagaaatccagatgtcatcaggaaacttactgttcacaggacatctcaagtgcaggctgaggatggacaaactacagctcaaaggaatgtcatactctatgtgcacaggaaagtttaaagttgtgaaggaaatagcagaaacacaacatggaacaatagttatcagagtacaatatgaaggggacggttctccatgtaagatcccttttgagataatggatttggaaaaaagacatgttttaggtcgcctgattacagtcaacccaatcgtaacagaaaaagatagcccagtcaacatagaagcagaacctccattcggagacagctacatcatcataggagtagagccgggacaattgaagctcaactggtttaagaaaggaagttctatcggccaaatgattgagacaacaatgaggggagcgaagagaatggccattttaggtgacacagcttgggattttggatccctgggaggagtgtttacatctataggaaaggctctccaccaagttttcggagcaatctatggggctgccttcagtggggtctcatggactatgaaaatactcataggagtcattatcacatggataggaatgaattcacgcagcacctcactgtctgtgtcactagtattggtgggagtcgtgacgctgtatttgggagttatggtgcaggcctga

SEQ ID NO: 5 - DNA, 96 bp, Artificial Sequence
  Description of Artificial Sequence Synthetic nucleotide construct
aaaaaggcgagaaatacgcctttcaatatgctgaaacgcgagagaggatccagcacctcactgtctgtgtcactagtattggtgggagtcgtgacg

SEQ ID NO: 6 - DNA, 56 bp, Artificial Sequence
  Description of Artificial Sequence Synthetic primer
tgcacattcgctctagaatttaggtgacactatagagttgttagtctacgtggacc

SEQ ID NO: 7 - DNA, 18 bp, Artificial Sequence
  Description of Artificial Sequence Synthetic primer
gcttcccggaggagtggc

SEQ ID NO: 8 - DNA, 18 bp, Artificial Sequence
  Description of Artificial Sequence Synthetic primer
gacccagcaagtatagcg

SEQ ID NO: 9 - DNA, 21 bp, Artificial Sequence
  Description of Artificial Sequence Synthetic primer
gacatggggatgggttcttca

SEQ ID NO: 10 - DNA, 39 bp, Artificial Sequence
  Description of Artificial Sequence Synthetic primer
acatgcttcgacctcgaggtggacctcggttgcggcaga

SEQ ID NO: 11 - DNA, 58 bp, Artificial Sequence
  Description of Artificial Sequence Synthetic primer
ctggaatcagctgagctcagaacctgttgattcaacagcaccattccattttctggcg

SEQ ID NO: 12 - DNA, 63 bp, Artificial Sequence
  Description of Artificial Sequence Synthetic primer
gcgagaaatacgcctttcaatatgctgaaacgcgagagaaacatgcatggagatacacctaca

SEQ ID NO: 13 - DNA, 42 bp, Artificial Sequence
  Description of Artificial Sequence Synthetic primer
tgcagttctcttttggtgcattggtttctgagaacagatggg

SEQ ID NO: 14 - DNA, 21 bp, Artificial Sequence
  Description of Artificial Sequence Synthetic primer
atgcaccaaaagagaactgca

SEQ ID NO: 15 - DNA, 36 bp, Artificial Sequence
  Description of Artificial Sequence Synthetic primer
aaggtcaaaattcaacagctgggtttctctacgtgt

SEQ ID NO: 16 - DNA, 72 bp, Artificial Sequence
  Description of Artificial Sequence Synthetic primer
atacagcgtcacgactcccaccaatactagtgacacagacagtgaggtgctggggccagggttggactcgac

SEQ ID NO: 17 - DNA, 63 bp, Artificial Sequence; identical to SEQ ID NO: 12
  Description of Artificial Sequence Synthetic primer

SEQ ID NO: 18 - DNA, 72 bp, Artificial Sequence; identical to SEQ ID NO: 16
  Description of Artificial Sequence Synthetic primer

SEQ ID NO: 19 - DNA, 36 bp, Artificial Sequence
  Description of Artificial Sequence Synthetic primer
atatccccgcggatgaataaccaacgaaaaaaggcg

SEQ ID NO: 20 - DNA, 35 bp, Artificial Sequence
  Description of Artificial Sequence Synthetic primer
atatatctagactaggcctgcaccataactcccaa

SEQ ID NO: 21 - DNA, 40 bp, Artificial Sequence
  Description of Artificial Sequence Synthetic primer
cctctagctagagcttaccatgaataaccaacgaaaaaag

SEQ ID NO: 22 - DNA, 35 bp, Artificial Sequence
  Description of Artificial Sequence Synthetic primer
gattagagctcttatctgcgtctcctgttcaagat

SEQ ID NO: 23 - DNA, 34 bp, Artificial Sequence
  Description of Artificial Sequence Synthetic primer
gcgctctagaatgactgcaggcatgatcattatg

SEQ ID NO: 24 - DNA, 46 bp, Artificial Sequence
  Description of Artificial Sequence Synthetic primer
atgccagtaggacaggtgtaatctaggcctgcaccataactcccaa

SEQ ID NO: 25 - DNA, 21 bp, Artificial Sequence
  Description of Artificial Sequence Synthetic primer
attacacctgtcctactggca

SEQ ID NO: 26 - DNA, 21 bp, Artificial Sequence
  Description of Artificial Sequence Synthetic primer
catggtaagctctagctagag

SEQ ID NO: 27 - DNA, 34 bp, Artificial Sequence; identical to SEQ ID NO: 23
  Description of Artificial Sequence Synthetic primer

SEQ ID NO: 28 - DNA, 35 bp, Artificial Sequence
  Description of Artificial Sequence Synthetic primer
gtatagagctcttatctgcgtctcctgttcaagat

SEQ ID NO: 29 - DNA, 595 bp, Cytomegalo virus
cattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaacc

SEQ ID NO: 30 - DNA, 84 bp, Hepatitis D virus
gggtcggcatggcatctccacctcctcgcggtccgacctgggcatccgaaggaggacgcacgtccactcggatggctaagggag

SEQ ID NO: 31 - DNA, 36 bp, Artificial Sequence
  Description of Artificial Sequence Synthetic primer
gcgcgcggtaccttgacattgattattgactagtta

SEQ ID NO: 32 - DNA, 42 bp, Artificial Sequence
  Description of Artificial Sequence Synthetic primer
ggtccacgtagactaacaactcggttcactaaacgagctctg

SEQ ID NO: 33 - DNA, 21 bp, Artificial Sequence
  Description of Artificial Sequence Synthetic primer
agttgttagtctacgtggacc

SEQ ID NO: 34 - DNA, 21 bp, Artificial Sequence
  Description of Artificial Sequence Synthetic primer
ccctgcagcattccaagtgag

SEQ ID NO: 35 - DNA, 42 bp, Artificial Sequence
  Description of Artificial Sequence Synthetic primer
ctcacttggaatgctgcagggcccaaggtgagatgaagctgt

SEQ ID NO: 36 - DNA, 42 bp, Artificial Sequence
  Description of Artificial Sequence Synthetic primer
gtggagatgccatgccgacccagaacctgttgattcaacagc

SEQ ID NO: 37 - DNA, 48 bp, Artificial Sequence
  Description of Artificial Sequence Synthetic primer
gggtcggcatggcatctccacctcctcgcggtccgacctgggcatccg

SEQ ID NO: 38 - DNA, 55 bp, Artificial Sequence
  Description of Artificial Sequence Synthetic primer
ctcccttagccatccgagtggacgtgcgtcctccttcggatgcccaggtcggacc

SEQ ID NO: 39 - DNA, 45 bp, Artificial Sequence
  Description of Artificial Sequence Synthetic primer
ccactcggatggctaagggagaataaaatgaggaaattgcatcgc

SEQ ID NO: 40 - DNA, 35 bp, Artificial Sequence
  Description of Artificial Sequence Synthetic primer
tatatccgcggatagaatgacacctactcagacaa

SEQ ID NO: 41 - DNA, 60 bp, Artificial Sequence
  Description of Artificial Sequence Synthetic primer
cagactgaaaaaagtattgaagacaatccagagatcgaaggaattaagaacaaccaaatc

SEQ ID NO: 42 - DNA, 36 bp, Artificial Sequence
  Description of Artificial Sequence Synthetic primer
ctccacattttccaagatttggttgttcttaattcc

SEQ ID NO: 43 - DNA, 75 bp, Artificial Sequence
  Description of Artificial Sequence Synthetic primer
cagactgaaaaaagtattgaagacaatccagagatcgaaggaattaagaacaaccaaatcttggaaaatgtggag

SEQ ID NO: 44 - protein, 5 aa, Artificial Sequence
  Description of Artificial Sequence Synthetic signal peptide
PQAQA

(19) United States
(12) Patent Application Publication
(10) Pub. No.: US 2009/0197319 A1
(43) Pub. Date: Aug. 6, 2009

(54) Virus-Like Particle Containing A Dengue Recombinant Replicon

(75) Inventor: Xiaowu Pang, Bethesda, MD (US)

Correspondence Address: MDIP LLC, POST OFFICE BOX 2630, MONTGOMERY VILLAGE, MD, 20886-2630, US

(73) Assignee: TenGen Biomedical Company, Rockville, MD (US)

(21) Appl. No.: 11/971,343

(22) Filed: Jan. 9, 2008

Related U.S. Application Data
(63) Continuation of application No. 11/194,342, filed on Aug. 1, 2005, now abandoned.
Application No. 11/194,342 is a continuation-in-part of application No. PCT/CN2004/000088, filed on Jan. 30, 2004.

(30) Foreign Application Priority Data
Jan. 30, 2003 (CN) CN03115272.4
Jan. 30, 2003 (CN) CN03115273.2

(51) Int. Cl. C12N 7/00; C12N 15/11

(52) U.S. Cl. 435/235.1; 536/23.72

(57) ABSTRACT

A diagnostic assay for detecting antibody, in part, to human papilloma virus includes a virus-like particle that expresses human papilloma virus antigen.

### FIELD OF THE INVENTION

The invention relates to a Dengue (DEN) vector and in particular to a recombinant virus that is prepared by replacing DEN structural genes with non-DEN transgenes, such as, proteins and antigens, for the recombinant production thereof, and the production and application.

### BACKGROUND

There is a constant need for new and useful cloning vectors for experimental use and for the development of scale up resources for the production of biological molecules in quantity. The flaviviruses are attractive candidates. Flaviviruses replicate cytoplasmically and therefore minimize interaction with the host genome, the genome can carry a large insert, the flaviviruses are easy to culture and maintain, expression is robust, the viruses do not inactivate the host and the genome is now being understood.

Thus, a flavivirus cloning vector can find utility in the laboratory for isolating genes, for maintaining and amplifying a gene, for gene delivery and so on.

Cancer continues to be a substantial threat to human health. Some cancers have been linked to a variety of viruses, most notably human papillomavirus (HPV), which can be responsible for cervical cancer. The prevalence of HPV infection of the female genital tract has a positive correlation with the pathological progression of the diseased cervix presenting in the normal progression of the disease beginning with chronic cervicitis→pseudocondyloma→verruca lesions→condyloma acuminate→cervical intraepithelial neoplasia→cervical cancers.

More than 75 percent of cervical cancers were found to be associated with HPV type 16 and/or HPV type 18 infection. Other types of HPV have been found to be associated with various forms of cancer in various populations. Those types include HPV, 30, 31, 39, 40, 45 and 59.

The current treatments for cervical cancer are surgery, radiotherapy, chemotherapy, and occasionally, such treatments are combined with therapies known as natural medicine, naturopathic medicine, herbal medicine, Chinese medicine and so on, using products or derivatives of natural products, such as plants, molds, fungi and the like. Unfortunately, the effect is not satisfactory, and the five-year survival rate is about 65 percent. The prognosis following the recurrence of disease is extremely poor, with only five percent survival over two years. The expense for cervical cancer screening and therapy in the USA is approximately 570 million dollars per year. Therefore, early detection is essential.

There are several new methods directed to treating cervical cancer including, use of inactivated vaccines, the earliest tumor vaccines with good safety, cannot induce effective cellular immune responses because the antigen generally cannot be expressed by a host cell; DNA virus vector vaccines, which to avoid oncogenicity, generally contains only parts of the oncogene, which in turn, reduces the immunogenicity of the antigen; Borysiewicz L K et al. (Lancet. 1996, Jun. 1:347(9014):1523-7) reported an HPV vaccine with vaccinia virus as vector; however, most people have antibody to vaccinia so the widespread use of such a vector is limited; WO01/53467 provides recombinant yellow fever virus (YFV) vectors, however, the recombinant YFV retained essentially the entire YFV genome without deletion of some relevant cis genes, and thus is replicable and infectious; and WO99/28487 provides an expression and delivery method of exogenous sequences by the flavivirus, kunjin virus (KUN); however, the titer of VLP was unsatisfactory and the booster immunization will be less effective because there is only one species of KUN and thus, repeated administration can lead to vector immunity.

The KUN genome is stable in plasmids which can be cloned and recombined with traditional methods. However, that strategy does not apply to other flaviviruses. For example, Dengue virus (DEN) sequences have lower stability in plasmids than KUN, probably because KUN belongs to the Japanese encephalitis virus group whereas DEN belongs to the different Dengue virus group. The two groups have only 45% homology at the level of the genome. So it is hard to construct DEN replicons and to obtain DEN VLPs directly following the teachings of WO99/28487.

WO02/072803 teaches a method for construction of DEN subgenomic replicons and use thereof for a DEN vaccine. Nevertheless, it is not possible to replicate those teachings.

In summary, there is an urgent requirement for novel cloning vectors with high capacity; and for host cells that are long-lived, can carry large inserts and are easy to propagate. Such vectors also are not oncogenic, and can be administered repeatedly.

### SUMMARY

The invention aims to provide novel products applicable to laboratory and industrial production of recombinant gene products which have good safety, high titer, good booster effects, and are versatile in what exogenous genes can be carried. That is achieved in the use of DEN replicons and virus-like particles.

In a first aspect the invention provides recombinant DEN replicons comprising deletion of one or more nucleotides including but not limited to, for example, the following nucleotide sequences:
a) coding region of the capsid protein, except for about the first 20 or so amino acids of the capsid protein;
b) the envelope protein coding region except for about the last 24 or so amino acids of the envelope protein, which also functions as the signal peptide of the first non-structural (NS1) protein; and
c) the preM region; wherein
exogenous nucleotide sequences (i.e., non-DEN), i.e. the transgene, the desired foreign gene to be expressed, generally is inserted between a) and b). The transgene can be about 3-4 kb in size, and from any source. A larger number of DEN structural gene nucleotides can be removed to accommodate a larger transgene. Mutation can also occur in regulatory regions and in sequences required for genome replication such as the 5′ untranslated region (UTR) and the 3′ UTR.

In preferred embodiments, the 3′ end of any exogenous nucleotide sequence contains 3′ release elements upstream of the coding region of the NS1 signal peptide, which can be the last about 24 amino acids of the envelope protein. Other signal peptides can be used as well. These release elements can be a nucleotide sequence obtained from the autoprotease of foot-and-mouth disease virus (FMDV) (SEQ ID NO:3), such as the about 20 amino acids thereof known in the art as 2A.

In another preferred embodiment, DEN is obtained from any one of the following: DEN type I, DEN type II, DEN type III or DEN type IV.

In another preferred embodiment, the coding region of the NS1 signal peptide is composed of the about last 72 nucleotides of the 5′ terminus of the E gene. Alternatively, a suitable signal peptide encoding sequence can be subcloned upstream in an operable situs of the NS1 coding sequence.

In another preferred embodiment, the 5′ untranslated region is followed by a sequence of about 60 nucleotides that are complementary and can base pair with the 3′ end of the replicon to enable circularization of the replicon for replication. In some embodiments, the about 60 nucleotides comprise the sequence encoding the first about 20 amino acids of the capsid protein.

In another preferred embodiment, the exogenous sequence encodes a human papilloma virus (HPV) antigen, an immunoregulator or both.

In yet another aspect, the present invention also provides virus-like particles (VLP) comprising a DEN recombinant replicon and deletion of one or more DEN structural proteins. Preferably, coding sequences of the capsid, membrane and envelope proteins are deleted.

In a further aspect, the present invention also provides cells for a packaging system for producing DEN recombinant replicons VLPs. These packaging cells are selected from:
(i) cells transfected by a plasmid with deleted structural genes of a DEN recombinant replicon,
(ii) cells transfected by a vector derived from a helper virus containing sequences of the structural proteins which are deleted from the DEN recombinant replicon (i), and
(iii) cells integrated with structural genes which are deleted from the DEN recombinant replicon (i) in the genome thereof,

and above cells express DEN structural proteins for complementing replicon packaging and the structural gene expression does not affect the growth of the host cells.

In another aspect, the present invention provides a method for production of a virus-like particle (VLP) as herein described comprising the steps of:
(i) introducing into a DEN packaging cell line, a DEN recombinant replicon or a VLP containing said replicon;
(ii) culturing that cell line either so it can express the DEN structural proteins; or introducing into that cell line replicons of a helper virus containing a DEN structural gene if the cell line cannot express that DEN structural gene, then culturing the cell line; and
(iii) collecting DEN-VLP containing flavivirus recombinant replicons.

In another preferred embodiment, said packaging cells contain a DEN structural protein expression vector, selected from a DEN vector with a mutated NS3 or a Tet-regulated vector.

In another embodiment, the replicon is used to produce the transgene expression product. That expression product can be used in diagnostic assay, such as positive controls. The expression product can be used as an immunogen for developing, for example, antibody thereto. Alternatively, the expression product can be used as a positive control in a diagnostic assay or as a binding agent for antibody in an immunoassay. The expression product can be configured for secretion into the culture medium, for expression on the VLP or for expression on a host cell.

Additional features and advantages are described herein, and will be apparent from, the following Detailed Description and the figures.

### BRIEF DESCRIPTION OF THE FIGURES

FIG. 1 shows preparation of a Dengue vector carrying HPV sequences.

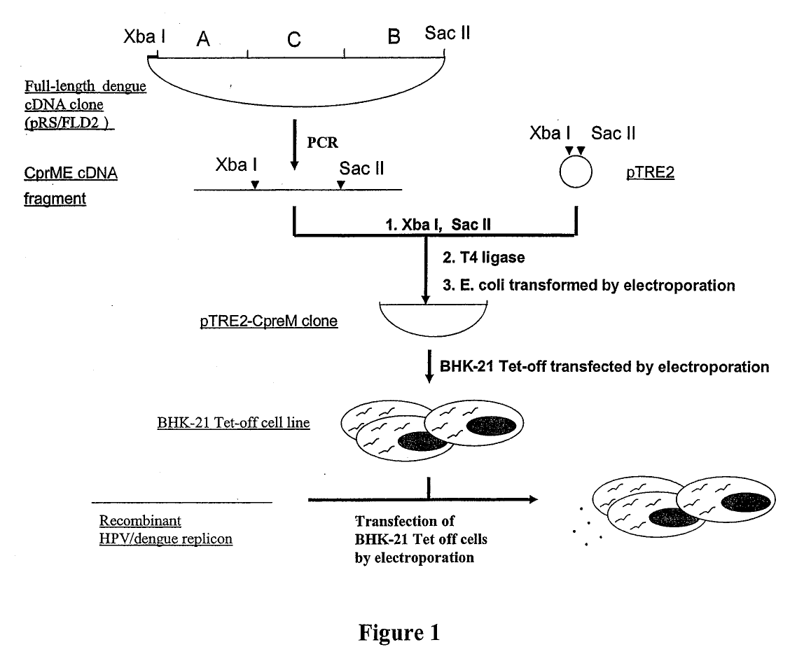

FIG. 2 shows an alternative preparation of a Dengue vector carrying HPV sequences by another method.

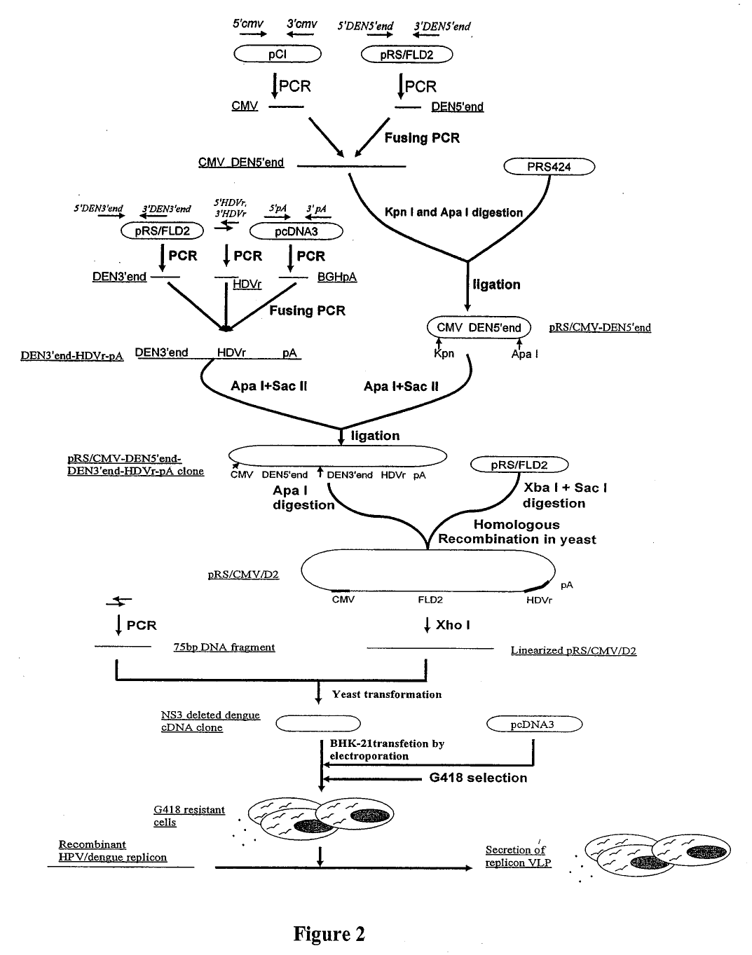

### DETAILED DESCRIPTION

The present invention relates to materials and methods for making a DEN-VLP preparation, as exemplified in the construction of a VLP carrying expressed HPV sequences with good safety, high titer and good booster effects.

“Nt” as used herein means a nucleotide.

“Release element” refers to nucleotide sequences at the 3′ end of a structural polypeptide coding sequence or at the 5′ end of the NS1 signal peptide coding region, used to enhance release of the polypeptide by a signal peptide protease without unrelated sequences after protein translation. A suitable release element is the about 60 nucleotide sequence of the Foot-And-Mouth virus hydrolytic enzyme, known as 2A, as provided in SEQ ID NO:3. Any nucleotide sequence that has the protease attracting properties of 2A can be used in a vector of interest. Usually there is only one “release element” per vector, but there may be plural “release elements” in a vector.

The terms “immunological activity” or “immunogenicity” refers to the ability to induce a specific humoral and/or cellular immunity of a mammal by natural, recombinant or synthetic immunogens, such as peptides.

The terms “antigen polypeptide” or “antigen peptide” refers to the amino acid sequence eliciting an immune response of a mammal whether single or combined with other helper molecules (for example, Human histocompatibility antigen (HLA) I or II).

The term “immune response” refers to a cellular and/or humoral immune response, for example, sufficient to inhibit or prevent, for example, infection or disease caused by microbes or to generate reagents that can be used in diagnostic assays, such as specific cytotoxic T cells, antibody and specific B cells.

The terms “object,” “individual” or “patient” refers to any object which needs diagnosis or therapy, especially mammals, such as human. Other objects include other mammals, such as, cow, dog, cat, cavy, rabbit, rat, mouse, horse etc.

A vector of interest will have one or more of the following characteristics to form a recombinant replicon vector that is disabled in the context of not containing an intact genome:
1. all of the non-structural gene sequences must be present in the vector. Any deletion of a structural gene or loss of expression of a structural gene must be one that does not have a negative impact on non-structural protein expression and does not result in a frame shift mutation of a non-structural protein and a signal peptide thereof.
2. the first 60 or so nt of the capsid (C) protein gene (the coding sequence of the first 20 amino acids of the C protein, that is, the amino terminus of the capsid protein) must be present in the vector because those sequences are complementary to the 3′-UTR sequences which form a circle for flavivirus gene replication and virus packaging. If that region is shorter than 60 nt, the efficiency of VLP formation will decrease. Sequences longer than 60 nt (for example, 81, 99, 120, 150 and 180 nt) can form VLP, however, the possible length of any exogenous gene will decrease accordingly because of packaging limitations. So the optimal dimension is retention of about the first 60-150 nt of the 5′ terminus of the C protein gene, optimally 60-120 nt. A synthetic sequence that is complementary to the 3′ end of the flavivirus genome also can be used, whether that 3′ terminus is the native, wild-type UTR or a synthetic UTR.
3. the NS1 signal peptide, namely the last 24 or so amino acids of the envelope (E) protein, must be present in the vector. The corresponding coding sequence is the last about 72 nts of the E protein gene. If shorter than 72 nts, the efficiency of VLP formation will decrease. If longer than 72 nt (for example, 81, 99, 120 or 150 nt), the vector can still form a VLP, however, the length of the exogenous gene insert will decrease accordingly because of packaging limitations. So the optimal dimension is retention of 72-150 nt, optimally 60-120 nt. Alternatively, synthetic or other signal peptides that are operable with NS1 can be used.
4. the deletion of a structural gene and insertion of an exogenous gene in the deleted site cannot impact NS1 signal peptide function, as well as function of the downstream non-structural genes. The DEN structural protein coding region includes CpreME (preM is a premembrane protein). Usually, portions of the CpreME coding sequence can be deleted (about 100-2000 bp, optimally 500-2000 bp). Alternatively, all structural protein coding sequences aside from the provisos noted above, can be deleted from a vector of interest.

The DEN virus that can used to make a vector in the present invention has no special limitation and can be any subtype of DEN virus. Four subtypes of DEN genome that can be used, for example, have the following ATCC accession numbers:

| | |
|---|---|
| DEN virus type I | M87512; |
| DEN virus type II | M29095; |
| DEN virus type III | M93130; and |
| DEN virus type IV | AF289029. |

DEN virus has the property of being bound by dendritic cells which can enhance immunogenicity effects. Furthermore, DEN has an ADE property (antibody dependent enhancement of infection) which refers to the observation that after a first immunization of DEN, antibody dependent infection is enhanced when the host is immunized with a different DEN subtype. So DEN is effective for repetitive, booster immunizations. The ADE phenomena can be used for exogenous protein expression systems, which not only avoids neutralization of vector caused by repetitive immunization, but also increases the efficiency of infection of vectors into cells and exogenous protein delivery efficiency. Because DEN has four subtypes, any one can be used for an exogenous expression vector, through further inoculation with different DEN recombinant replicons, that is, of a different subtype, tends to intensify immunization efficiency.

Thus, the expression product of a DEN vector can be a useful immunogen for generating host antibody, for example, to obtain antiserum to the expressed transgene, because any host vector immunity that might occur can be overcome by delivering the transgene using a vector made from a different DEN subtype.

Also, the expression product of a DEN vector can be a useful absorbent of antibody directed to the expressed transgene. Accordingly, an expression product of a DEN vector can be used as a capture reagent in a solid phase assay for detecting antibody to the expressed transgene, the expression product of a DEN vector substituting for the capture antibody often used in such immunoassays. The expression product of a DEN vector is affixed to a solid phase, for example, a membrane, a surface, such as a plastic surface, a bead and the like, in a variety of formats, such as, in the case of a membrane, a strip, a sheet and the like. A sample suspected of containing antibodies to the transgene expressed by the DEN vector is exposed to the affixed DEN vector, the solid phase is washed as needed and as known in the art, and then exposed to a suitable reporter molecule that will reveal antibody bound to the solid phase. For example, the reporter can be an antibody, a receptor and the like, with specificity for antibody. The reporter is a molecule that is detectable and thus contains a label, such as a radioisotope, a fluorescent compound, a structure visible to the naked eye or with minimal magnification, such as a colored bead, such as a liposome containing a dye, a metal sol and so on.

The expression product of a DEN vector can be secreted and collected in the culture medium as known in the art. Alternatively, the expression product can be obtained from the host cells by lysis of the cells and purification, practicing methods known in the art. The artisan can also configure the vector and sequences contained therein for expression of the transgene product at the surface of the VLP. In that case, the VLP itself will substitute for the transgene expression product. Also, the expression product can be expressed at the surface of a host cell containing said DEN VLP. In that case, the host cell can substitute for the transgene expression product.

Exogenous genes used in the present invention have no special limitation. It can be any exogenous coding sequence, such as an aptamer, siRNA, polypeptide, ribozyme and the like, any therapeutic gene, such as a gene encoding an antibody to VEGF, an interferon, a cytokine and so on, a tumor antigen gene, a virus antigen gene, a microbe antigen gene, an immunoregulator gene and so on. The representative antigens include (but are not limited to): human HPV antigen (such as the E6 or E7 protein of the 16 and 18 subtypes), HIV antigen, HBV antigen, HCV antigen, EBV antigen, HTLV-1 antigen, MAGE, a Salmonella antigen, BCG, a Streptococcus antigen, an H. influenza antigen, an S. pneumoniae antigen, BAGE, CAGE etc. The length of an exogenous gene has no special limitation, usually the foreign coding sequence is from about 100 bp to about 2000 bp, optimally from about 150 bp to about 1200 bp. Any size of transgene can be used so long as the DEN genome can be packaged into a particle. Furthermore, a release element, such as 2A, can be added at the 3′ end of an exogenous gene to ensure a high level of expression of the expressed sequence. If the intercalated exogenous gene is a tumor antigen gene or a virus antigen gene, said VLP can be used for prevention and therapy of tumor and virus diseases.

Taking human papilloma virus (HPV) as an example, the present invention provides diagnostic, preventative and therapeutic compositions against diseases caused by HPV infection. Therein said composition contains a VLP packaged using a DEN recombinant replicon using necessary complementing DEN structural proteins. Therein said exogenous nucleic acid sequence of the DEN recombinant replicon encodes an antigen of one or many HPV subtypes. The vector can contain also a sequence encoding an immunoregulator, or the immunoregulator sequence may be carried by a second vector. Therein said HPV antigen protein can be E6, E7 or an E6/E7 fusion or composite molecule oncoprotein of, for example, HPV type 16 or HPV type 18, or can be an HPV major capsid protein L1 or minor capsid protein L2. Therein said immunoregulator can be a gene sequence encoding a polypeptide with an immunoregulating activity obtained from, for example, IL2, IL12, IL18, GM-CSF etc. or a functional portion thereof.

The composition provided by said invention has the advantage of being relatively non-immunogenic to the host, and thus can be administered repeatedly with effect. Such an anti-human papilloma virus composition is a useful immunogen, and provides prevention and therapy for diseases caused by papilloma virus, such as chronic cervicitis, pseudocondyloma, verrucous lesions, condyloma acuminata, cervical intraepithelial neoplasia, cervical cancer etc., is useful in diagnostic assays and so on.

Construction of said replicon can be processed as follows:
(1) construction of a full-length DEN genome sequence, this can be fulfilled by normal PCR and ligation techniques as known in the art;
(2) Deletion of parts of one or more DEN structural genes, this can be fulfilled by homologous recombination or synthesis of a deleted sequence that is cloned into and replaces the portion to be deleted from the DEN genome, a known method is homologous recombination in yeast; and
(3) Insertion of an exogenous gene coding sequence, though the exogenous gene can be inserted into regions with partial structural gene deletion by a known method, a preferred method is homologous recombination in yeast where sequences homologous to two sites flanking the insertion site are ligated to the two ends of the exogenous gene, introducing said construct into yeast along with a DEN genome DNA with a structural gene deletion, then obtaining the replicon carrying the exogenous gene inserted therein through homologous recombination, the recombination incidence approaches 100%, making construction relatively easy, controllable and stable.

After obtaining the replicon, it can be introduced into packaging cells to produce VLP. A normal method is after the DEN replicon is introduced into cells, then the helper virus expressing a necessary structural protein is introduced into those cells. Another method is construction of expression packaging cells with an inducible or constitutive plasmid carrying the DEN structural gene as a vector and then introduction of the replicon (or VLP) into these cells to produce VLP. For example, constitutive packaging cells can be made that do not express a functional NS3 gene. The NS3 gene possesses a special packaging signal sequence and thus, a DEN genome with an NS3 deletion can't be packaged into a VLP. So if the packaging cell doesn't have a functional DEN NS3 gene, it can't be packaged into VLP themselves. VLP produced by this packaging cell only contains a DEN recombinant replicon, and thus, there is no need to screen amongst the replicons for those that are recombinant.

Specifically, such methods include:
(a) the packaging cells can contain tetracycline-regulated (or other regulatable methods) gene expression boxes for expressing a DEN structural protein. The tet regulated system is known in the art and includes tet operators, tet repressors, and transgene expression is induced by exposure to tetracycline;
(b) after a recombinant DEN replicon is introduced into cells and cultured (for example, about 24 hrs), a helper virus, for example, an alphavirus replicon, expressing a complementing DEN structural protein (or other expression vector) is introduced into said cells to produce VLP containing DEN replicon;
(c) if the packaging cell expresses a DEN genome with partial reduction of NS3 gene expression, a recombinant DEN replicon is introduced into said packaging cell to produce a VLP containing DEN replicon; and
(d) after infected with a VLP and cultured, cells are infected by a helper virus, such as an alphavirus replicon, expressing a complementing DEN structural protein (or other nonreplicable virus vector) to produce a VLP containing a DEN replicon.

The present invention also provides various compositions comprising such a recombinant DEN replicon and/or a VLP, including a pharmaceutical composition.

Various compositions comprising such a recombinant DEN can include different buffers according to practical purposes and substances suitable to other purposes required of a pharmaceutical delivery form, as known in the art. These compositions generally contain pharmaceutically available carriers, diluents and excipients as known in the pharmaceutics arts, “Remington: Pharmacology and Pharmacological Practice” 19^{th} ed. (1995) Mack Publishing Co.

Pharmaceutical compositions can be made into a variety of forms, such as for injection, solid forms, such as grains, tablets, pills, suppositories and capsules, other liquid forms, such as a suspension, a spray etc. For example, some of the known carriers include water for injection, plant and animal oil and fats, and so on, as known in the art. The stabilizer, wetting agent, emulsifier, salt suitable for controlling osmolarity, various buffers suitable for maintenance of appropriate pH, surfactants, permeants to enhance transdermal movement and so on can be used for auxiliary materials as known in the art.

The said recombinant DEN can be put into dispensing means as known in the art. Thus, for example, if administered by injection or intravenously, the VLP can be suspended in a suitable medium or can be presented in a desiccated form, for example, by cryopreservation for reconstitution prior to use. For such desiccation, the preparation likely will include suitable excipients to facilitate the process, such as a cryoprotectant, such as glycerol, as known in the art.

Moreover, the said compositions can also contain other components such as an adjuvant, a stabilizer, a pH regulator, a preservative etc. These components are familiar to technicians of this field. Adjuvants include but are not limited to an aluminum adjuvant, a saponin adjuvant, a Ribi adjuvant (Ribi ImmunoChem Research Inc., Hamilton, Mont.), a Montanide ISA adjuvant (Seppic, Paris, France), a Hunter's TiterMax adjuvant (CytRx Corp., Norcross, Ga.), a Gerbu adjuvant (Gerbu Biotechnik GmbH, Gaiberg, Germany) and so on. In addition, other components for regulating and modifying the immunological response can be used.

The said recombinant DEN replicon and/or VLP can be administered to an individual through known methods. The said composition is often administered through a normal administering pathway or modeling pathogen infection pathway for a biologic. Pharmaceutically available vehicles can be used when administrating oral compositions such as flavorants, colorants, enteric coatings etc.

Normal and pharmaceutically available administration pathways include intranasal, intramuscular, intratracheal, subcutaneous, intracutaneous, endovaginal, intrapulmonary, intravenous, nasal, oral or other extraintestinal pathways. Combined administration can be made if needed or it can be regulated according to the transgene, the disease, the disease condition and so on. The vaccine composition can be administered as a single dose or in multiple doses, and also contain a booster dose to elicit and/or to maintain immunity.

“Effective dose” is given in an amount such that the availability of DEN recombinant replicon and/or VLP is of an amount so as to elicit an immune response and effectively prevent a host against a virus infection, tumor or other source of pathology etc. Usually, after infecting host cells, every dose of vaccine is enough to produce from about 1 to about 1000 ug, or about 1 to about 200 ug, or about 10 to about 100 ug of transgene. The vaccine effective dose calculated with recombinant DEN nucleic acids as a basis usually includes administrating about 1 to about 1000 ug nucleic acids. Furthermore, the average range of vaccine effective dose is about 10² to about 10⁹, about 10³ to about 10⁷, or about 10⁴ to about 10⁵ plaque forming units (PFU). The optimal dose of vaccine can be determined, for example, by antibody titer of experimental objects and standard investigation methods of other reactions, as known in the art. Whether a booster dose is needed can be detected by supervising immunity levels using immune assays as known in the art. After evaluating serum antibody titer, one or more booster doses can be administered. Administering adjuvant and/or immunological stimulant can enhance an immune response to the target transgene.

The vector of interest can be configured into a kit for diagnostic use, relating to the transgene contained therein. The kit can contain the expressed transgene, the VLP expressing the transgene or a cell expressing the transgene affixed on a surface usable for the assay. The kit also can contain a suitable reporter system for detecting the specific analyte of interest. The reporter can be, for example, an antibody, a receptor, a ligand and the like that has a specific binding function. The reporter will contain a detectable marker, such as a bead, a metal sol, a nanoparticle, a Quantum Dot, a fluorescent molecule and so on. A preferred detectable marker is one that can be detected with a minimum of intervention. Thus, one that is visible to the naked eye is desirable. The kit will include instructions suitable for making use of the replicon of interest.

Compared with known technology, the present invention has the following advantages:

1. enhancement of immunological effectiveness;
(1) The dendritic cell is the most effective antigen presenting cell. In the present invention, antigen is effectively expressed and presented in said infected cells eliciting an effective immune response against antigen with a DEN replicon as vector, capitalizing on the affinity of DEN for dendritic cells because of the DEN receptor on dendritic cells.
(2) Utilizing the characteristic of different DEN, repetitive infection and the ADE phenomenon, the user can inoculate repetitively to enhance immunological effects.
(3) As tumor antigen and virus antigen, an E6 and/or E7 recombinant DEN replicon can express E6 and/or E7 antigen in cells for an extended time and effectively can stimulate immunologic reaction to antigen for an extended period.

2. enhancement of vaccine safety;
(1) Recombinant DEN replicon RNAs replicate and are expressed exclusive in the cytoplasm, and thus, it is difficult for recombination to occur in the somatic cell genome.
(2) The recombinant DEN replicon is a recombinant DEN RNA with a structural gene deletion. That replicon only replicates and expresses in somatic cells and doesn't form infectious virus particles.
(3) The immune system has the ability to recognize and clear recombinant replicon. As a result, it isn't permanently present in vivo.

3. little treatment distress; and
(1) Administration just a few times, reducing patient distress in the therapy process.
(2) The composition operates quickly in the host, thereby shortening the course of therapy.

4. low cost:
(1) The packaging cells expressing a DEN structural protein are transfected by a DEN recombinant replicon to produce VLP and then are transfected by VLP again to produce substantial quantities of VLP, increasing VLP construction efficiency. That avoids massive replicon construction, shortens vaccine production, increases vaccine production efficiency and decreases cost.
(2) The constitutive expressing packaging cells without NS3 expression are transfected by a DEN recombinant replicon to produce VLP. NS3 has a special packaging signal sequence and a DEN genome with an NS3 deletion can't be packaged into a VLP. So, packaging cells can't package and form VLP themselves. VLP produced by this packaging cell contains recombinant DEN replicon only, and there is no need to screen virus particles for recombinants, thereby enhancing construction efficiency.

The invention now is exemplified in the following representative examples. Clearly, these examples only clarify and do not limit this invention. The experimental methods taught herein can be performed practicing such known methods, such as those taught in Sambrook et al., Molecular Cloning: A Laboratory Manual (New York, Cold Spring Harbor Laboratory Press, 1989) or recommended by manufacturers.

### Example 1

### Construction of Full-length DEN Virus cDNA Clones

1. Full-length DEN cDNA clones were constructed by incorporation of full-length DEN cDNA into plasmid pRS424, wherein said DEN II (NGC) (ATCC#VR-1255) virus and plasmid pRS424 (ATCC#77105) were obtained from the ATCC, Manassas, Va.
   a) Three DEN cDNA fragments (3′-, 5′- and middle cDNA) were produced by PCR with DEN RNA as template. 5′-cDNA contains an XbaI restriction site and an Sp6 enhancer sequence at the 5′ end. The 3′-cDNA contains a SacI restriction site at the 3′ end. The middle cDNA contains sequences identical to the 3′ end of the 5′-cDNA and the 5′ end of the 3′-cDNA.

Sequences of Primers:

| | | |
|---|---|---|
| | | SEQ ID |
| Primer | Sequence(5′-3′) | NO: |

| | | |
|---|---|---|
| 1a. | TGCACATTCGCTCTAGAATTTAGGTGACACTATAGAG | 6 |
| | TTGTTAGCTACGTGGACC | |
| 1b. | GCTTCCCGGAGGAGTGGC | 7 |
| 2a. | GACCCAGCAAGTATAGCG | 8 |
| 2b. | GACATGGGGATGGGTTCTTCA | 9 |
| 3a. | ACATGCTTCGACCTCGAGGTGGACCTCGGTTGCGGCA | 10 |
| | GA | |
| 3b. | CTGGAATCAGCTGAGCTCAGAACCTGTTGATTCAACA | 11 |
| | GCACCATTCCATTTTCTGGCG | |

The resulting three DEN cDNA fragments are 5484 bp, 2530 bp and 2922 bp, respectively, in length.
   b) The 3′ cDNA and the 5′ cDNA fragments were ligated into plasmid pRS424 practicing standard methods.
   c) Using known methods of homologous recombination in yeast, the middle cDNA fragment was inserted into the said clone to produce a full-length DEN cDNA clone, pRS/FLD2.
2. Construction of DEN replicon cDNA clones with structural protein gene sequences deleted
   a) Therein said full-length DEN cDNA clone pRS/FLD2 produced in step 1(c) was linearized by BamHI digestion at position 1696 or 2203 of the DEN cDNA sequence (GenBank Accession No. AF038403) (There is a BamHI restriction site in DEN genome).
   b) A 96 nt DNA fragment was synthesized by fusion PCR containing 45 bases deriving from codon 6 to codon 20 of the structural protein at the 5′ end, 45 bases deriving from codon 751 to codon 766 of the structural protein at the 3′ end and the BamHI restriction site in the middle. The sequence of the DNA is provided as in SEQ ID NO:5.
   c) The linearized pRS/FLD2, 96 nt cDNA fragment and activated yeast were put into yeast transforming buffer (PEG buffer) and incubated at 30° C. for one hour, then the linearized pRS/FLD2 and 96 nt cDNA fragment can be transformed into yeast automatically. After culture for two days, the transformed yeast colonies can be seen. Based on homologous recombination in yeast, the 96 nt cDNA fragment can replace structural protein sequences (SEQ ID NO:4) to produce DEN replicon cDNA clones with structural protein sequences deleted. Like recombinant HPV-DEN RNA replicons produced in Example 3 below, a DEN RNA replicon without structural protein sequences can be used to produce DEN RNA replicon virus-like particles (VLP) by at least four different packaging schemes as provided hereinabove, and will be described in Example 4 below.

### Example 2

### Construction of HPV E7-E6-2A DNA Fragment

1) E7-E6 sequence is an HPV oncogene. (GenBank Accession No. AF486352; AF469197; and AF472508) designated as SEQ ID NO:1, which encodes an antigen containing 225 amino acids (SEQ ID NO:2).
2) Therein said 2A sequence is a mouth-foot-pestilence virus fragment containing sixty bases, designated as SEQ ID NO:3.
3) E6 and E7 fragments were produced by PCR with plasmid HPV-16 as template, oligos GCGAGAAATACGCCTTTCAATATGCTGAAACGCGAGAGAAACATGC ATGGAGATACACCTACA (SEQ ID NO: 12) and TGCAGTTCTCTTTTGGTGCATTGGTTTCTGAGAACAGATGGG (SEQ ID NO: 13) as one set of primers, oligos ATGCACCAAAAGAGAACTGCA (SEQ ID NO: 14) and AAGGTCAAAATTCAACAGCTGGGTTTCTCTACGTGT (SEQ ID NO: 15) as another set of primers. The 5′ end of the E6 fragment contains the sequence, ATGCACCAAAAGAGAACTGCA which is identical to the 3′ end of the E7 fragment. The 3′ end of the E6 fragment contains the sequence, CAGCTGTTGAATTTTGACCTT which is identical to the 5′ end of the 2A sequence. The 5′ end of the E7 fragment contains a sequence identical to DEN II cDNA nt 108-124. A 111 bp DNA fragment containing the 2A sequence and its 3′ end sequence identical to DEN II cDNA nt 2275-2375 was produced by fusion PCR with oligos CAGCTGTTGAATTTTGACCTTCTTAAGCTTGCGGGAGACGTCGAGTC CAACCCTGGCCCC (SEQ ID NO: 3) and ATACAGCGTCACGACTCCCACCAATACTAGTGACACAGACAGTGAG GTGCTGGGGCCAGGGTTGGACTCGAC (SEQ ID NO: 16) as primers. 4) Full-length E7-E6-2A cDNA fragment was produced by PCR with the E6 fragment, the E7 fragment and the 111 bp fragment containing the 2A sequence as template, oligos GCGAGAAATACGCCTTTCAATATGCTGAAACGCGAGAGAAACATGC ATGGAGATACACCTACA (SEQ ID NO: 17) and ATACAGCGTCACGACTCCCACCAATACTAGTGACACAGACAGTGAG GTGCTGGGGCCAGGGTTGGACTCGAC (SEQ ID NO:18) as primers. The 5′ end and 3′ end of the construct contains sequences identical to DEN cDNA gene.

### Example 3

### Integration of HPV Genome into DEN RNA Replicon

1) Full-length DEN cDNA clone pRS/FLD2) produced in step 1 (c) of Example 1 was linearized with BamHI.
2) The linearized DEN cDNA clone (pRS/FLD2) and HPV E7-E6-2A cDNA fragment were transformed into yeast together. In the process of yeast DNA replication, the HPV-E7-E6-2A cDNA fragment can integrate into the DEN cDNA clone automatically and replace the DEN structural protein sequences (C, M and E coding region sequences) to produce a circular HPV-DEN RNA replicon cDNA clone (pRS/D2-HPV16).
3) The HPV-DEN RNA replicon cDNA clone produced in step 2 was purified with a Qiagen (Qiagen Inc.) column.
4) The HPV-DEN RNA replicon cDNA clone was transformed into Stab 12™ (Invitrogen Inc., Carlsbad, Calif.) and a large number of HPV-DEN RNA replicon cDNA clones were produced by this system.
5) This HPV-DEN RNA replicon cDNA clone was digested by SacI at the 3′ end.
6) The modified HPV-DEN RNA replicon cDNA was transcribed into RNA by DNA dependent Sp6 RNA polymerase to produce a recombinant HPV-DEN RNA replicon.
7) The recombinant HPV-DEN RNA replicon was transfected into BHK-21 (ATCC#CCL-10) cells by electroporation and recombinant HPV-DEN RNA replicons replicate and express E7-E6 protein of HPV.

### Example 4

### Four Ways of Production of HPV VLP

### Method 1: Production of VLP Using Tetracycline-Regulated Gene Expression System

Summary: Using the tetracycline-regulated gene expression system, BHK-21 cells were transformed to produce a DEN structural protein regulated expression cell line. The Tet-Off Gene Expression systems were purchased from Clontech Inc. This plasmid expresses a regulatory protein named “rtTA” which regulates gene expression and plasmid transcription. This gene expression system contains pTet-Off regulatory vectors, pTRE2 response vectors and pTK-Hyg selection vectors.
1) DEN structural protein genes (full 2328 bp DEN structural protein genes designated as SEQ ID NO:4) were inserted into pTRE2 response vectors of the Tet-Off gene expression system. Namely the CpreME cDNA fragment was produced by PCR with full-length DEN cDNA clone (pRS/FLD2) as template, oligos ATATCCCCGCGGATGAATAACCAACGAAAAAAGGCG (SEQ ID NO:19) and ATATATCTAGACTAGGCCTGCACCATAACTCCCAA (SEQ ID NO:20) as primers. The CpreME cDNA fragment and pTRE2 (Clontech Inc.) were digested by XbaI and Sac II. After Qiagen spin column (Qiagen Inc.) purification, these two fragments were ligated by T4 ligase (New England Biolab Inc.) and used to transform E. coli to produce recombinant pTRE2 containing DEN structural protein genes.
2) Transform BHK-21 cells with the pTet-Off regulatory vectors by electroporation and then BHK-21 Tet-Off cell lines are selected.
3) The Tet-Off gene expression system inserted with the structural protein genes produced in step 1 was transformed into BHK-21 Tet-Off cells by electroporation to produce a DEN structural proteins regulated-high expression BHK-21 cell line.
4) The recombinant HPV-DEN RNA replicon produced in Example 3 was transformed into said BHK-21 cell line by electroporation. After one day, the Tet-Off gene expression system was induced for high expression of structural proteins. After ten days, the supernatant was collected to produce 10 packaged recombinant HPV-DEN RNA replicon VLP per ml.

### Method 2: Production of HPV Recombinants Using Sindbis RNA Replicon

1) Production of DEN Structural Protein Fragment:
   a) A DNA fragment of a C protein gene was produced by PCR with pRS/FLD2 produced in Step 1(c) of Example 1 as template, and oligos CCTCTAGCTAGAGCTTACCATGAATAACCAACGAAAAAAG (SEQ ID NO:21) and GATTAGAGCTCTTATCTGCGTCTCCTGTTCAAGAT (SEQ ID NO:22) as primers.
   b) A DNA fragment of prM-E gene was produced by PCR with pRS/FLD2 produced in Step 1(c) of Example 1 as template, and oligos GCGCTCTAGAATGACTGCAGGCATGATCATTATG (SEQ ID NO:23) and ATGCCAGTAGGACAGGTGTAATCTAGGCCTGCACCATAACTCCCAA (SEQ ID NO: 24) as primers.
   c) A DNA fragment of Sindbis 26S was produced by PCR with Sindbis RNA replicon cDNA (Stratagene Co.) as template, and oligos ATTACACCTGTCCTACTGGCA (SEQ ID NO:25) and CATGGTAAGCTCTAGCTAGAG (SEQ ID NO:26) as primers.
   d) The 3′ end of the prM-E DNA fragment and the 5′ end of the Sindbis 26S DNA fragment contain an identical 21 nt DNA sequence. The 3′ end of the Sindbis 26S DNA fragment and the 5′ end of the C gene DNA fragment contain an identical 21 nt DNA sequence.
   e) The C/preM-E fragment and the Sindbis 26S DNA fragment were fused together by PCR with the three fragments as templates, and oligos GCGCTCTAGAATGACTGCAGGCATGATCATTATG (SEQ ID NO:27) and GTATAGAGCTCTTATCTGCGTCTCCTGTTCAAGAT (SEQ ID NO:28) as primers to produce DNA fragment preME-26S-C.
2) Production of recombinant Sindbis RNA replicon containing DEN structural protein genes.
   a) The PreME-26S-C fragment produced in step 1 and the Sindbis RNA replicon DNA plasmid (Stratagene Co.) were digested by XbaI and SacI. After Qiagen spin column (Qiagen Inc.) purification, these two DNA fragments were ligated by T4 ligase (New England Biolab Inc.) and used to transform E. coli to produce recombinant Sindbis RNA replicon cDNA clones containing DEN structural protein genes and then was purified by Qiagen spin column (QIAGEN Inc.)
   b) Recombinant Sindbis RNA replicon cDNA clone produced in step (a) was linearized by XhoI.
   c) Linearized Sindbis RNA replicon cDNA clone was transcribed into RNA by DNA dependent Sp6 RNA polymerase to produce a recombinant Sindbis RNA replicon containing DEN structural protein genes.
3) Recombinant HPV-DEN RNA replicon produced in Example 3 was transfected into BHK-21 cells. After 24 hours, recombinant Sindbis RNA replicons produced in said step 2(c) were transfected into the cells again. The two transfections were done by electroporation with a 0.4 cm Gene Pulser Cuvette, 200 V/950 uF. After one or two days, the supernatant was collected to produce 10⁴-10⁵ recombinant HPV-DEN RNA replicon VLP per ml.

### Method 3: Production of Particles Using Sindbis RNA Replicon VLP.

1) Recombinant Sindbis RNA replicons produced in step 2(c) of Method 2 and DH-BB RNA (Stratagene Inc.) were transfected into BHK-21 cells (ATCC#CCL-10) together by electroporation. After three to five days, the supernatant was collected to obtain recombinant Sindbis RNA replicon VLP containing DEN structural protein genes.
2) Recombinant HPV-DEN RNA replicon VLPs produced in step 3 of Method 2 were infected into BHK-21 cells (ATCC#CCL-10). After 24 hours, recombinant Sindbis RNA replicon VLPs produced in said step 1 were infected into the cells again. After 24 to 48 hours, the supernatant was collected to obtain 10⁵-10⁶ recombinant HPV-DEN RNA replicon VLPs per ml.
   Method 4: Production of BHK-21 Packaging Cell Line Transformed with DEN cDNA Sequence Deleted NS3 Region
1. A DNA fragment of cytomegalovirus (CMV) containing the immediate-early enhancer/promoter region was produced by PCR with pCI (Promega Co.) as template, oligos 5′ CMV and 3′ CMV as primers. As an early enhancer and promoter, this DNA fragment (SEQ ID NO:29) of CMV is 631 bp in length. The DNA fragment of the 5′ end of DEN cDNA (DEN 5′ end) was produced by PCR with pRS/FLD2 produced in Step 1(c) of Example 1 as template, and oligos 5′ DEN 5′ end and 3′ DEN 5′ end as primers. These two DNA fragments were fused together by fusion PCR with these two fragments as template, and oligos 5′ CMV and 3′ DEN 5′ end as primers to produce the DNA fragment, CMV-DEN 5′ end (CMV-DEN 5′ end).
2. The CMV-DEN 5′ end fragment produced in Step 1 and plasmid pRS424 (ATCC#77105) were digested by KpnI and ApaI. After Qiagen spin column (Qiagen Inc.) purification, these two DNA fragments were ligated by T4 ligase (New England Biolab Inc.) and used to transform E. coli to produce CMV-DEN 5′ end clone (pRS/CMV-DEN 5′ end).
3. The DNA fragment of the 3′ end of DEN cDNA (DEN 3′ end) was produced by PCR with pRS/FLD2 produced in Step 1(c) of Example 1 as template, and oligo 5′ DEN 3′ end and 3′ DEN 3′ end as primers. A DNA fragment of the hepatitis delta virus antigenomic ribozyme (HDVr, SEQ ID NO:30) was produced by PCR with oligo 5′ HDVr and 3′ HDVr as primers. A DNA fragment of bovine growth hormone poly A (BGH pA) was produced by primers. The three DNA fragments were fused together by fusion PCR with the three fragments as templates, and oligos 5′ DEN 3′ end and 3′ pA as primers to produce DNA fragment, DEN 3′ end-HDVr-pA.

Sequences of primers:

| | | |
|---|---|---|
| | | SEQ |
| | | ID |
| Primers | Sequence(5′-3′) | NO: |

| | | |
|---|---|---|
| 5′CMV | GCGCGCGGTACCTTGACATTGATTATTGACTAGTTA | 31 |
| 3′CMV | GGTCCACGTAGACTAACAACTCGGTTCACTAAAC | 32 |
| | GAGCTCTG | |
| 5′DEN5′end | AGTTGTTAGTCTACGTGGACC | 33 |
| 3′DEN5′end | CCCTGCAGCATTCCAAGTGAG | 34 |
| 5′DEN3′end | CTCACTTGGAATGCTGCAGGGCCCAAGGTGAGATG | 35 |
| | AAGCTGT | |
| 3′DEN3′end | GTGGAGATGCCATGCCGACCCAGAACCTGTTGATT | 36 |
| | CAACAGC | |
| 5′HDVr | GGGTCGGCATGGCATCTCCACCTCCTCGCGGTCCGA | 37 |
| | CCTGGGCATCCG | |
| 3′HDVr | CTCCCTTAGCCATCCGAGTGGACGTGCGTCCTCCTT | 38 |
| | CGGATGCCCAGGTCGGACC | |
| 5′pA | CCACTCGGATGGCTAAGGGAGAATAAAATGAGGAAA | 39 |
| | TTGCATCGC | |
| 3′pA | TATATCCGCGGATAGAATGACACCTACTCAGACAA | 40 |

4. Plasmid pRS/CMV-DEN 5′ end produced in step 2 and DNA fragment DEN 3′ end-HDVr-pA produced in step 3 were digested by ApaI and SacII. After Qiagen spin column (Qiagen Inc.) purification, these two DNA fragments were ligated by T4 ligase (New England Biolab Inc.), transformed into E. coli and selected to produce pRS/CMV-DEN 5′ end-DEN 3′ end-HDVr-pA plasmid DNA.
5. Plasmid DNA pRS/CMV-DEN 5′ end-DEN 3′ end-HDVr-pA produced in step 4 was digested by ApaI and plasmid pRS/FLD2 produced in step 1(c) of Example 1 was digested by XbaI and SacI. The two digestive products were purified, used to transform yeast and selected to produce full-length DEN RNA replicon cDNA clone (pRS/CMV/D2) according to homologous recombination in yeast.
6. Full-length DEN RNA replicon cDNA clone (pRS/CMV/D2) produced in step 5 was digested by XhoI to produce linearized full-length DEN RNA replicon cDNA clone. A 75 bp DNA fragment (CAGACTGAAAAAAGTATTGAAGACAATCCAGAGATCGAAGGAATT AAGAACAACCAAATCTTGGAAAATGTGGAG, SEQ ID NO:43) was produced by PCR with oligos CAGACTGAAAAAAGTATTGAAGACAATCCAGAGATCGAAGGAATT AAGAACAACCAAATC (SEQ ID NO:41) and CTCCACATTTTCCAAGATTTGGTTGTTCTTAATTCC (SEQ ID NO:42) as primers. The linearized DEN RNA replicon cDNA clone and 75 bp DNA fragment were used to transform yeast resulting in a construct where a part of the NS3 gene (nt 5059-6215) was deleted in the linearized DEN RNA replicon cDNA by homologous recombination in yeast.
7. The NS3-deleted DEN cDNA clone produced in step 6 and pcDNA3 (Invitrogen, Inc.) were transfected into BHK-21 cells (ATCC#CCL-10) together. After culture for one day, these cells were changed to culture medium containing G418 (Sigma). A stable G418-resistant packaging cell line was selected by transfection of recombinant HPV-DEN RNA replicon produced in Example 3 into G418-resistant cells. The titer of recombinant HPV-DEN RNA replicon VLP can reach 10⁶.

### Example 5

### Animal Experiments of VLP-HPV Immunogenicity

To examine the immunity of an HPV expressing VLP, the HPV VLP was used to determine any effect on tumor cells using C57BL/6 mice.

C57BL/6 mice were inoculated with JHU-1 HPV cells as the tumor model, JHU-1 HPV cells contained E6 and E7 oncogenes of HPV. Eight-week-old mice were inoculated in the flank with 500 μl PBS buffer containing 105 JHU-1 HPV cells. JHU-1 HPV cells can completely induce solid tumors. Seven days after inoculation of JHU-1 HPV cells, tumors could be felt. After fourteen days, the tumors could reach over 6 mm in diameter.

Rx of HPV VLP: Four groups of eight C57BL/6 mice

| | flank inoculation | | | |
|---|---|---|---|---|
| | day 1 | Day 14 | Day 21 | Day 35 |
| group 1 | | | | HPV E6/E7-specific |
| control | | | | CD⁸⁺ T cell assay |
| group 2 | 1 × 10⁵ JHU-1 | 10⁷ PFU VLP | 10⁷ PFU VLP | |
| tumor control | Cells | | | |
| group 3 | 1 × 10⁵ JHU-1 | 10⁷ PFU | 10⁷ PFU | |
| VLP treatment | Cells | HPV-VLP | HPV-VLP | |
| group 4 | 500 μl PBS | 10⁷ PFU | 10⁷ PFU | HPV E6/E7-specific |
| VLP control | | HPV-VLP | HPV-VLP | CD⁸⁺ T cell assay |

PFU = Infectious Particle of HPV replicon-containing VLP
Groups 2 and 3 of eight mice each were inoculated in the flank with 10⁵ JHU-1 HPV cells. After fourteen days, groups 3 and 4 of eight mice each were inoculated ip with 10⁷ PFU HPV-VLP and after seven days they were inoculated with 10⁷ PFU HPV-VLP again.

1. To examine immunity of HPV VLP:
a) After inoculation of mice with JHU-1 HPV cells, tumor growth and sizes were examined every two days.
b) An HPV E6/E7-specific CD⁸⁺ T cell assay was used to examine the cells by cytoplasm staining and flow cytometry. After the second inoculation of group 4 mice with HPV-VLP, spleen cells were separated and used for staining with fluorescence-IFNγ and TNF-α antibodies. Stained cell surface IFN and TNF can be detected with flow cytometry.

2. Results:
a) The effects of HPV VLP on tumor growth

group 2 (tumor control)

| flank inoculation | mouse 1 | mouse 2 | mouse 3 | mouse 4 | mouse 5 | mouse 6 | mouse 7 | mouse 8 |
|---|---|---|---|---|---|---|---|---|
| of JHU-1 Cells (day) | tumor(mm) | tumor(mm) | tumor(mm) | tumor(mm) | tumor(mm) | tumor(mm) | tumor(mm) | tumor(mm) |
| Day 14 | 6 | 6 | 7 | 7 | 7 | 8 | 8 | 8 |
| Day 21 | 12 | 14 | 15 | 15 | 16 | 16 | 17 | 17 |
| Day 28 | 15 | 16 | 17 | 18 | 18 | 18 | 19 | 20 |
| Day 35 | 19 | 22 | >25* | >25 | >25 | >25 | >25 | >25 |

If a tumor is larger than 25 mm, the mouse was sacrificed.

group 3 (VLP treatment)

| flank inoculation | mouse 1 | mouse 2 | mouse 3 | mouse 4 | mouse 5 | mouse 6 | mouse 7 | mouse 8 |
|---|---|---|---|---|---|---|---|---|
| of JHU-1 Cells (day) | tumor(mm) | tumor(mm) | tumor(mm) | tumor(mm) | tumor(mm) | tumor(mm) | tumor(mm) | tumor(mm) |
| Day 14 | 6 | 7 | 7 | 7 | 7 | 8 | 8 | 8 |
| Day 21 | 3 | 5 | 5 | 6 | 6 | 7 | 9 | 10 |
| Day 28 | 0 | 0 | 0 | 0 | 2 | 4 | 8 | 10 |
| Day 35 | 0 | 0 | 0 | 0 | 0 | 4 | 9 | 11 |

In group 3, after inoculation of C57BL/6 mice with 10⁵ JHU-HPV for fourteen days, tumors were induced, as expected. The tumor bumps sometimes were larger than 6 mm in diameter. Then those mice were inoculated ip with 10⁷ HPV-VLP. After two days it can be seen that most tumors began to shrink. After first inoculation, after seven days, the mice were inoculated with 10⁷ HPV-VLP again. After two inoculations for one week, tumors in almost half the mice disappeared. In other mice, the trend was for diminution of tumor size. In group 2, after inoculation of C57BL/6 mice with 10⁵ JHU-HPV for thirty-five days, tumors of 75% of the mice were larger than 25 mm.
b) The effects of HPV VLP on E6-E7-specific CD⁸⁺ T cells

| | IFNγ | TNFα |
|---|---|---|
| group 1 control | 0.04% | 0.08% |
| group 4 VLP control | 1.5% | 1.28% |

The effects of the HPV VLP on E6-E7-specific CD⁸⁺ T cells were examined by cytoplasm staining. The HPV VLP induced CD⁸⁺ T cells to secrete IFNγ and TNFα.

### Example 6

### Construction of Different Types of DEN II VLP Vectors

A recombinant DEN II replicon was constructed as in Examples 1, 2 and 3 as described, but the difference was that the sequence of the 5′ end of the C gene and the length of the NS1 signal peptide were different. VLP were produced as in Example 4 described above.

Results:

| retained | retained | | | |
|---|---|---|---|---|
| C gene | E gene | | replication and | |
| fragment | fragment | inserted sequence | expression | Packaging |
| 30 | 72 | no | no | No |
| 60 | 45 | HPV16 E7-E6-2A | no | No |
| 120 | 72 | HPV16 E7-E6-2A | yes | Yes |
| 15 | 30 | HPV16 E7-E6-2A | no | No |
| 90 | 120 | HPV16 E7-E6-2A | yes | Yes |
| 90 | 150 | HPV16 E7-E6-2A | yes | Yes |
| 120 | 72 | HPV16 E7-E6 | no | No |

### Example 7

### Construction of DEN I VLP

A recombinant DEN replicon was constructed as in Examples 1, 2 and 3 above, but the difference is that DEN I replaced DEN II of Examples 1, 2 and 3. Then VLP were produced by method 4 of Example 4. Four mice of Example 5 with tumors were inoculated as group 3 of Example 5 described above.

All publications are herein incorporated by reference to the same extent as if each individual publication was specifically and individually indicated to be incorporated by reference in its entirety.

It will occur to those of ordinary skill in the art that various modifications may be made to the disclosed embodiments and that such modifications are intended to be within the scope of the present invention, which is defined by the following claims.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A recombinant dengue replicon expressing a transgene.

2. The replicon of claim 1, wherein said transgene encodes an antigen.

3. The replicon of claim 2, wherein said antigen is from a microbe or a virus.

4. The replicon of claim 3, wherein said virus is human papilloma virus (HPV).

5. The replicon of claim 1 wherein said dengue is subtype I.

6. The replicon of claim 1 wherein said dengue is subtype II.

7. The replicon of claim 1 wherein said dengue is subtype III.

8. The replicon of claim 1 wherein said dengue is subtype IV.

9. The replicon of claim 2, wherein said antigen is associated with cancer.

10. The replicon of claim 4, wherein said HPV is type 16.

11. The replicon of claim 4, wherein said HPV is type 18.

12. A virus-like particle (VLP) comprising the replicon of claim 1.

13. A kit comprising the replicon of claim 1 and instructions.

14. The kit of claim 13, further comprising a reporter.

15. The kit of claim 14, wherein said reporter comprises an antibody.

16. The kit of claim 15, wherein said reporter comprises a visible marker.
